(19) United States
(12) Patent Application Publication
(10) Pub. No.: US 2021/0315971 A1
(43) Pub. Date: Oct. 14, 2021

(54) COMPOSITIONS ACTING AS PRO(RENIN) RECEPTOR ANTAGONISTS FOR THE TREATMENT OF NON-ALCOHOLIC FATTY LIVER DISEASE

(71) Applicant: Board of Regents of the Nevada System of Higher Education on behalf of the University of Nevada,Reno, Reno, NV (US)

(72) Inventor: Yumei Feng EARLEY, Reno, NV (US)

(21) Appl. No.: 17/265,497

(22) PCT Filed: Aug. 6, 2019
(86) PCT No.: PCT/US2019/045362

§ 371 Date: Feb. 2, 2021

Related U.S. Application Data
(60) Provisional application No. 62/715,717, filed on Aug. 7, 2018.

(51) Int. Cl. A61K 38/17; A61K 45/06; A61P 3/10; A61P 1/16

(52) U.S. Cl. CPC A61K 38/1796; A61P 1/16; A61P 3/10; A61K 45/06

(57) ABSTRACT

Disclosed are methods of treating metabolic diseases, such as non-alcoholic fatty liver disease (NAFLD), including nonalcoholic steatohepatitis (NASH), and Type II diabetes. In some examples, the method includes administration of an effective amount of a (pro)renin receptor (PRR) antagonist to a subject in need thereof, such as a patient at risk of acquiring or afflicted with NAFLD, NASH and/or Type II diabetes, to prevent, inhibit, and/or reduce one or more signs or symptoms associated with NAFLD, NASH and/or Type II diabetes, such as by reducing the severity of liver steatosis.

### CROSS REFERENCE TO RELATED APPLICATION

This disclosure claims the benefit of the earlier filing date of U.S. Provisional Application No. 62/715,717, filed Aug. 7, 2018, which is specifically incorporated by reference herein in its entirety.

### ACKNOWLEDGEMENT OF GOVERNMENT SUPPORT

This invention was made with government support under R01 HL122770 awarded by the National Institutes of Health. The government has certain rights in the invention.

### FIELD

This disclosure relates to the fields of metabolic diseases and in particular, to use of (pro)renin receptor (PRR) antagonists for treating non-alcoholic fatty liver disease (NAFLD), such as nonalcoholic steatohepatitis (NASH), and Type II diabetes.

### BACKGROUND

In the United States, non-alcoholic fatty liver disease (NAFLD) is the most common liver disease and associated with higher mortality according to data from earlier National Health and Nutrition Examination Survey. It is estimated that 20% of the US population has NAFLD. Diet and physical exercise are considered the first line of treatment for patients with NAFLD, but their results on therapeutic efficacy are often contrasting. Behavior therapy is necessary most of the time to achieve a sufficient result. Pharmacological therapy includes a wide variety of classes of molecules with different therapeutic targets and, often, little evidence supporting the real efficacy. Despite the abundance of clinical trials, NAFLD therapy remains a challenge for the scientific community, and there are no licensed therapies for NAFLD.

### SUMMARY

(Pro)renin receptor (PRR) is a new component of the renin-angiotensin system (RAS). The inventor discovered that the knockdown of the PRR in the central nervous system attenuated the development of high-fat diet induced liver steatosis in mice. The mechanisms include regulating lipid metabolism in the liver and modulating the autonomic control of the liver through the brain. Furthermore, subcutaneous administration of PRO20 attenuates the high-fat diet induced liver steatosis. It is believed that PRR antagonism reduces NAFLD through dual functions: acting on the central nervous system and directly acting on the liver.

Based upon these findings, disclosed are methods of treating metabolic diseases, such as NAFLD, such as NASH, including administration of an effective amount of a PRR antagonist to a subject in need thereof, such as a patient at risk of acquiring or afflicted with liver steatosis, to prevent, inhibit, and/or reduce one or more signs or symptoms associated with NAFLD, such as by reducing the severity of liver steatosis by at least 50%. In some examples, the PRR antagonist is one or more antagonists disclosed in U.S. Pat. Nos. 9,573,976 and 9,586,995, each of which is hereby incorporated by reference in its entirety. In some examples, the PRR antagonist is PRO20. In some examples, the disclosed methods attenuate or reverse the development of NAFLD, including NASH. In some examples, the disclosed methods are used to treat NAFLD with Type II diabetes or Type II diabetes alone.

The foregoing and other features of the disclosure will become more apparent from the following detailed description, which proceeds with reference to the accompanying figures.

### BRIEF DESCRIPTION OF THE DRAWINGS

FIG. 1 is a schematic illustrating PRR signal transduction pathways.

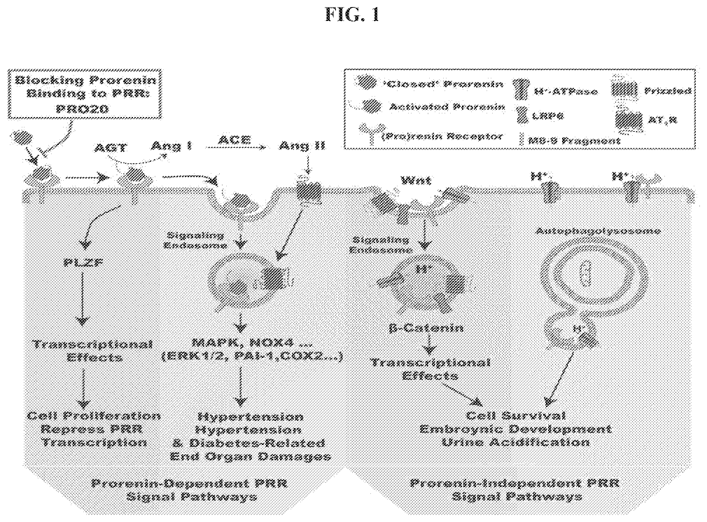

FIG. 2 demonstrates PRR deletion in the neurons reduces high fat diet-induced type 2 diabetes.

FIG. 3 demonstrates PRR deletion in the neurons prevents high fat diet-induced islet hypertrophy in 6 weeks.

FIG. 4A demonstrates PRR deletion in the neurons reduces high fat diet-induced liver steatosis.

FIG. 4B demonstrates PRR deletion in the neurons reduces high fat diet-induced hypertension.

FIG. 5 demonstrates PRR deletion in the neurons modulate liver lipogenesis and lipolysis pathways.

FIG. 6 illustrates subcutaneous administration of PRR antagonist, PRO20, attenuates high fat diet induced-liver steatosis in mice.

FIG. 7 illustrates subcutaneous administration of PRR antagonist, PRO20, attenuates peroxisome proliferator-activated receptor gamma (PPARγ). PPARγ is a key nuclear receptor signaling responsible for promoting liver lipid storage, expression of lipogenic genes and is usually increased upon high fat diet. PRO20 treatment prevents the upregulating of PPARγ in the liver supporting a mechanism for the beneficial role of PRO20 in preventing fatty liver development.

FIG. 8 illustrates subcutaneous administration of PRR antagonist, PRO20, prevents activation of liver renin-angiotensin system induced by high fat diet.

FIG. 9 is a schematic illustrating PRR inhibition for the treatment of NASH: mechanisms of action.

### DETAILED DESCRIPTION OF SEVERAL EMBODIMENTS

### Terms

The following explanations of terms and methods are provided to better describe the present disclosure and to guide those of ordinary skill in the art in the practice of the present disclosure. The singular terms “a,” “an,” and “the” include plural referents unless context clearly indicates otherwise. Similarly, the word “or” is intended to include “and” unless the context clearly indicates otherwise. The term “comprises” means “includes.” Thus, “comprising A or B,” means “including A, B, or A and B,” without excluding additional elements. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

It is further to be understood that all base sizes or amino acid sizes, and all molecular weight or molecular mass values, given for nucleic acids or polypeptides are approximate, and are provided for description. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below.

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless indicated otherwise, the nomenclature of substituents that are not explicitly defined herein are arrived at by naming the terminal portion of the functionality followed by the adjacent functionality toward the point of attachment.

A person of ordinary skill in the art would recognize that the above definitions are not intended to include impermissible substitution patterns (e.g., methyl substituted with 5 different groups, pentavalent carbon, and the like). Such impermissible substitution patterns are easily recognized by a person of ordinary skill in the art.

All publications, patent applications, patents, and other references mentioned herein are incorporated by reference in their entirety. In case of conflict, the present specification, including explanations of terms, will control. In addition, the materials, methods, and examples are illustrative only and not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a PRR antagonist is disclosed and discussed and a number of modifications that can be made are discussed, each and every combination and permutation of the PRR antagonist and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

In order to facilitate review of the various embodiments of this disclosure, the following explanations of specific terms are provided:

Administration: To provide or give a subject one or more agents, such as a PRR antagonist and/or treats one or more symptoms associated with NAFLD, such as NASH, by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

Effective amount: An amount of agent that is sufficient to generate a desired response, such as reducing or inhibiting one or more signs or symptoms associated with a condition or disease. When administered to a subject, a dosage will generally be used that will achieve target tissue/cell concentrations. In some examples, an “effective amount” is one that treats one or more symptoms and/or underlying causes of any of a disorder or disease. In some examples, an “effective amount” is a therapeutically effective amount in which the agent alone with an additional therapeutic agent(s) (for example, an agent for NAFLD and/or NASH), induces the desired response such as treatment of a metabolic disorder, such as NAFLD, including NASH.

In particular examples, it is an amount of a PRR antagonist capable of decreasing, inhibiting or reducing PRR activity by least 20%, at least 50%, at least 60%, at least 70%, at least 80%, at least 90%, at least 95%, at least 98%, or even at least 100%, such as by 20% to 95%, 30% to 95% and the like. In some examples, it is an amount of a PRR antagonist capable of reducing the severity of liver steatosis by at least 50%, at least 60%, at least 70%, at least 80%, at least 90%, at least 95%, at least 98%, or even at least 100%, such as between about 50% and about 70%, about 50% and about 90%, about 50% and about 95%, about 70% and 95%, about 90% and 98%, about 80% and 95% and the like.

In some examples, an effective amount is an amount of a pharmaceutical preparation that alone, or together with a pharmaceutically acceptable carrier or one or more additional therapeutic agents, induces the desired response.

In one example, a desired response is to increase the subject's survival time by slowing the progression of the disease. The disease does not need to be completely inhibited for the pharmaceutical preparation to be effective. For example, a pharmaceutical preparation can decrease the progression of the disease by a desired amount, for example by at least 20%, at least 50%, at least 60%, at least 70%, at least 80%, at least 90%, at least 95%, at least 98%, or even at least 100%, as compared to the progression typical in the absence of the pharmaceutical preparation.

In another or additional example, it is an amount sufficient to partially or completely alleviate symptoms of the NASH within the subject. Treatment can involve only slowing the progression of the disease temporarily, but can also include halting or reversing the progression of the disease permanently.

Effective amounts of the agents described herein can be determined in many different ways, such as assaying for a reduction in of one or more signs or symptoms associated with the NAFLD, such as NASH, in the subject or measuring the expression level of one or more molecules known to be associated with NAFLD and/or NASH. Effective amounts also can be determined through various in vitro, in vivo or in situ assays, including the assays described herein.

The disclosed agents can be administered in a single dose, or in several doses, for example daily, during a course of treatment. However, the effective amount can be dependent on the source applied, the subject being treated, the severity and type of the condition being treated, and the manner of administration.

The phrase “therapeutically effective amount” means an amount of a therapeutic, prophylactic, and/or diagnostic agent (e.g., composition comprising a PRR antagonist) that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition.

Inhibiting a disease or condition: A phrase referring to reducing the development of a disease or condition, for example, in a subject who is at risk for a disease or who has a particular disease. Particular methods of the present disclosure provide methods of inhibiting NAFLD, NASH and/or Type II Diabetes.

Nonalcoholic fatty liver disease (NAFLD): A condition in which fat builds up in the liver. Nonalcoholic steatohepatitis (NASH) is a type of NAFLD.

Optional or optionally: An event, circumstance, or material that may or may not occur or be present, and that the description includes instances where the event, circumstance, or material occurs or is present and instances where it does not occur or is not present.

Polypeptide or peptide: A polymer in which the monomers are amino acid residues which are joined together through amide bonds. When the amino acids are alpha-amino acids, either the L-optical isomer or the D-optical isomer can be used. The terms “polypeptide,” “peptide” or “protein” as used herein are intended to encompass any amino acid sequence and include modified sequences such as glycoproteins. The terms “polypeptide” and “peptide” are specifically intended to cover naturally occurring proteins, as well as those which are recombinantly or synthetically produced. The term “residue” or “amino acid residue” includes reference to an amino acid that is incorporated into a protein, polypeptide, or peptide.

A conservative substitution in a polypeptide is a substitution of one amino acid residue in a protein sequence for a different amino acid residue having similar biochemical properties. Typically, conservative substitutions have little to no impact on the activity of a resulting polypeptide. For example, a protein or peptide including one or more conservative substitutions (for example no more than 1, 2, 3, 4 or 5 substitutions) retains the structure and function of the wild-type protein or peptide. A polypeptide can be produced to contain one or more conservative substitutions by manipulating the nucleotide sequence that encodes that polypeptide using, for example, standard procedures such as site-directed mutagenesis or PCR. In one example, such variants can be readily selected by testing antibody cross-reactivity or its ability to induce an immune response. Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

(Pro)renin receptor antagonist (PRR antagonist): A composition able to antagonize the action of prorenin. PRR antagonists can bind to the (pro)renin receptor and prevent or block (pro)renin from binding. Alternatively, PRR antagonists can bind to (pro)renin and prevent or block (pro) renin from binding to the (pro)renin receptor.

Recombinant: A recombinant nucleic acid or protein is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques. The term recombinant includes nucleic acids and proteins that have been altered solely by addition, substitution, or deletion of a portion of a natural nucleic acid molecule or protein.

Subject: Living multi-cellular vertebrate organisms, a category that includes human and non-human mammals. The term subject can be used interchangeably with “individual” or “patient.”

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Calculations of sequence similarity or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In an embodiment, the percent identity between two amino acid sequences is determined using the Needleman and Wunsch, (1970, J. Mol. Biol. 48: 444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package, using a NWS gap DNA. CMP matrix and a gap weight of 40, 50, 60, 70, or 80 and a length weight of 1, 2, 3, 4, 5, or 6. In one embodiment, a set of parameters (and the one that should be used unless otherwise specified) are a Blossum 62 scoring matrix with a gap penalty of 12, a gap extend penalty of 4, and a frameshift gap penalty of 5.

Treatment: To partially or completely alleviating, ameliorating, relieving, delaying onset of, inhibiting progression of, reducing severity of, and/or reducing incidence of one or more symptoms or features of a particular disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Under conditions sufficient for: A phrase that is used to describe any environment that permits the desired activity. In one example, includes administering a disclosed agent to a subject sufficient to allow the desired activity.

### METHODS OF USE

Methods of Treating Metabolic Diseases

Disclosed are methods of treating a metabolic disease, including NAFLD, such as NASH, and/or Type II diabetes comprising administering to a subject an effective amount of a composition comprising a PRR antagonist to a subject in need thereof. In some example, the method of treatment prevents, inhibits or reduces one or more signs or symptoms associated with the metabolic disease in the subject.

In particular, methods of treating NAFLD, such as NASH, are disclosed. In some instances, the PRR antagonist is a polypeptide, such as a polypeptide disclosed in U.S. Pat. Nos. 9,573,976 and 9,586,995, each of which is hereby incorporated by reference in its entirety.

Disclosed are methods of treating NAFLD, including NASH, comprising administering to a subject a therapeutically effective amount of a composition comprising a PRR antagonist, wherein the PRR antagonist is a polypeptide, wherein the polypeptide comprises an amino acid sequence having at least 70%, such as at 75%, at least 80%, at least 85%, at least 90%, at least 95%, at least 98%, such as between 70% and 98%, 75% and 95%, 80% and 98%, 85% and 98%, 90% and 98%, 90% and 95%, 95% and 98%, 98% and 100%, such as 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity to the amino acid sequence of PRO20 set forth in SEQ ID NO:2 (LPTDTTTFKRIFLKRMPSI). U.S. Pat. Nos. 9,573,976 and 9,586,995 refer to PRO20 as PR20. The two are the same antagonist with an amino acid sequence set forth in SEQ ID NO. 2 and therefore, the two names can be used interchangeably herein. In some instances, the polypeptide comprises an amino acid sequence having at least at least 70%, such as at 75%, at least 80%, at least 85%, at least 90%, at least 95%, at least 98%, such as between 70% and 98%, 75% and 95%, 80% and 98%, 85% and 98%, 90% and 98%, 90% and 95%, 95% and 98%, 98% and 100%, such as 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity to the amino acid sequence set forth in SEQ ID NO:1.

Disclosed are methods of treating NAFLD, including NASH comprising administering to a subject a therapeutically effective amount of a composition comprising a PRR antagonist, wherein the PRR antagonist is a polypeptide, wherein the polypeptide comprises the amino acid sequence set forth in SEQ ID NO:1. Disclosed are methods of treating NAFLD, including NASH, comprising administering to a subject a therapeutically effective amount of a composition comprising a PRR antagonist, wherein the PRR antagonist is a polypeptide, wherein the polypeptide is PRO20 with the amino acid sequence set forth in SEQ ID NO:2.

Disclosed are methods of treating NAFLD, including NASH, comprising administering to a subject a therapeutically effective amount of a composition comprising a PRR antagonist, wherein the PRR antagonist is a polypeptide, wherein the polypeptide comprises the amino acid sequence XXTDXTTXXXXXXXXXXSX (SEQ ID NO:1).

Disclosed are methods of treating NAFLD, including NASH, comprising administering to a subject a therapeutically effective amount of a composition comprising a PRR antagonist, wherein the PRR antagonist is a polypeptide, wherein the polypeptide is the amino acid sequence XXTDXTTXXXXXXXXXXSX (SEQ ID NO:1). Each of the X's in SEQ ID NO:1 can be any amino acid. SEQ ID NO:1 is provided as an example of a polypeptide to be used in the methods described herein, wherein the sequence comprises 100% identity at amino acids 3, 4, 6, 7, and 18 to amino acids 3, 4, 6, 7, and 18 of SEQ ID NO:2.

Disclosed are methods of treating NAFLD, including NASH, comprising administering to a subject a therapeutically effective amount of a composition comprising a PRR antagonist, wherein the PRR antagonist is a polypeptide, wherein the polypeptide comprises the amino acid sequence XXTDXTTFXRIXXXXXXSX (SEQ ID NO:3).

Disclosed are methods of treating NAFLD, including NASH, comprising administering to a subject a therapeutically effective amount of a composition comprising a PRR antagonist, wherein the PRR antagonist is a polypeptide, wherein the polypeptide is the amino acid sequence XXTDXTTFXRIXXXXXXSX (SEQ ID NO:3). Each of the X's in SEQ ID NO:3 can be any amino acid. SEQ ID NO:3 is provided as an example of a polypeptide to be used in the methods described herein, wherein the sequence comprises 100% identity at amino acids 3, 4, 6, 7, 8, 10, 11, and 18 to amino acids 3, 4, 6, 7, 8, 10, 11, and 18 of SEQ ID NO:2.

Disclosed are methods of treating NAFLD, including NASH, comprising administering to a subject a therapeutically effective amount of a composition comprising a PRR antagonist, wherein the PRR antagonist is a polypeptide, wherein the polypeptide consists of the amino acid sequence of SEQ ID NO:2. Disclosed are methods of treating NAFLD, including NASH, comprising administering to a subject a therapeutically effective amount of a composition comprising a PRR antagonist, wherein the PRR antagonist is a polypeptide, wherein the polypeptide is the amino acid sequence of SEQ ID NO:2.

Disclosed are methods of treating NAFLD, including NASH, comprising administering to a subject a therapeutically effective amount of a composition comprising a PRR antagonist, wherein the composition further comprises a pharmaceutically acceptable carrier. Disclosed are methods of treating NAFLD, including NASH, comprising administering to a subject a therapeutically effective amount of a composition comprising a PRR antagonist, wherein the PRR antagonist is one or more of the polypeptides described herein, wherein the composition further comprises a pharmaceutically acceptable carrier.

1. Subject

In some examples, the subject is one at risk of acquiring or afflicted with NAFLD, such as NASH. In some examples, the subject is obese and/or has Type II Diabetes. In some examples, the subject does not have Type II Diabetes and/or hypertension. In some examples, the subject has NAFLD, but not Type II Diabetes. In some examples, the subject does not suffer from hypertension. In some examples, the subject has NAFLD, but does not suffer from hypertension. In some examples, the subject has NAFLD, but does not suffer from hypertension or Type II Diabetes. In some examples, the subject has NASH. In some examples, the subject has NASH, but not Type II Diabetes. In some examples, the subject does not suffer from hypertension. In some examples, the subject has NASH, but does not suffer from hypertension. In some examples, the subject has NASH, but does not suffer from hypertension or Type II Diabetes.

In some examples, the subject is a human.

2. Selecting and/or Diagnosing a Subject

In additional aspects, the method involves selecting a subject with a metabolic disease, such as NAFLD, including NASH, and/or Type II Diabetes. In some example, a subject is selected for treatment following diagnosing the subject with the metabolic disease. For example, the method can include diagnosing the subject as suffering from NAFLD, including NASH, and/or Type II Diabetes.

Methods of diagnosing a subject with NAFLD, including NASH, and/or Type II Diabetes, are known to those of skill in the art and include, but are not limited to, ultrasound, serum enzyme levels and/or liver biopsy. These methods are known to those of skill in the art.

In some examples, following the assay results, findings, diagnoses, predictions and/or treatment recommendations are recorded and communicated to technicians, physicians and/or patients, for example. In certain embodiments, computers are used to communicate such information to interested parties, such as, patients and/or the attending physicians. The therapy selected for administered is then based upon these results.

In one embodiment, the results and/or related information is communicated to the subject by the subject's treating physician. Alternatively, the results may be communicated directly to a test subject by any means of communication, including writing, such as by providing a written report, electronic forms of communication, such as email, or telephone. Communication may be facilitated by use of a computer, such as in case of email communications. In certain embodiments, the communication containing results of a diagnostic test and/or conclusions drawn from and/or treatment recommendations based on the test, may be generated and delivered automatically to the subject using a combination of computer hardware and software which will be familiar to artisans skilled in telecommunications. One example of a healthcare-oriented communications system is described in U.S. Pat. No. 6,283,761; however, the present disclosure is not limited to methods which utilize this particular communications system. In certain embodiments of the methods of the disclosure, all or some of the method steps, including the assaying of samples, diagnosing of diseases, and communicating of assay results or diagnoses, may be carried out in diverse (e.g., foreign) jurisdictions.

In several embodiments, identification of a subject as having NAFLD, such as NASH, results in the physician treating the subject, such as prescribing one or more disclosed PRR agents for inhibiting or delaying one or more signs and symptoms associated with NAFLD, such as NASH. In additional embodiments, the dose or dosing regimen is modified based on the information obtained using the methods disclosed herein.

3. NAFLD and NASH

Nonalcoholic fatty liver disease is a condition in which fat builds up in the liver. Non-alcoholic fatty liver disease is defined as the presence of macrovascular steatosis in the presence of less than 20 gm of alcohol ingestion per day. Nonalcoholic steatohepatitis (NASH) is a type of NAFLD. NAFLD, including NASH, can be determined by ultrasound, computed tomography (CT), magnetic resonance imaging (MRI), serum enzyme analysis and histological analysis of liver biopsy samples. For example, the characteristic ultrasonic feature is the “bright” liver with increased parenchymal echo texture and vascular blurring. In selected patients with NAFLD, measurement of liver stiffness by transient elastography has a diagnostic performance for fibrosis. For histology analysis, a histologic scoring systems such as that provided by the Pathology Committee of the NASH Clinical Research Network can be used.

4. Combination Therapy

Disclosed are methods of treating metabolic diseases, such as NAFLD, including NASH, and/or Type II Diabetes, comprising administering to a subject at risk of acquiring or afflicted with a metabolic disease an effective amount of a composition comprising a PRR antagonist, further comprising administering an additional known metabolic disease treatment, thereby decreasing one or more signs or symptoms associated with the metabolic disease. In some embodiments, the metabolic disease is NAFLD, such as NASH. In some embodiments, the PRR antagonist is a polypeptide. Known treatments for NAFLD, such as NASH, can be, but are not limited to, pharmacologic therapies such as statins, antihypertensive agents, antidiabetic drugs for patients with concurrent metabolic disorders, bariatric surgery, diet and/or exercise. While these are known treatments, as there are no approved medications for NASH, such treatments are still experimental.

In some instances, the PRR antagonist can be administered in conjunction with or followed by any of the known metabolic disease treatments, such as a known treatments for NAFLD, including NASH. In some instances, the PRR antagonist can be administered prior to the known metabolic disease treatments, such as a known treatment for NAFLD, including NASH. In some instances, the known metabolic disease treatments, such as a known treatment for NAFLD, including NASH, can be administered prior to the PRR antagonist. Administration of the PRR antagonist and known metabolic disease treatments, such as a known treatments for NAFLD, including NASH, can occur within minutes of each other, such as within about 5, 10, 15, 20, 25, 30, 40, 45, 50, 55, or 60 minutes of each other. In some instances, the administration of the PRR antagonist and known metabolic disease treatments, such as a known treatment for NAFLD, including NASH, can occur within hours of each other, such as 1, 2, 3, 4, 5, 6, 7, 8, 9, 10, 12, 14, 16, 18, 20, 22, or 24 hours of each other. In some instances, the administration of the PRR antagonist and known metabolic disease treatments, such as a known treatment for NAFLD, including NASH, can occur within days of each other, such as 1, 2, 3, 4, 5, 6, 7, 8, 9, 10, 12, 14, 16, 18, 20, 22, or 24 days or greater of each other.

5. (Pro)Renin Receptor Antagonists

As described herein, a PRR antagonist inhibits or blocks the ligand-receptor interaction of prorenin to PRR. PRR antagonists can block prorenin from binding PRR. In some instances, a PRR antagonist competes with prorenin for binding to PRR.

i. Polypeptides

PRR antagonists can be polypeptides. Examples of functional PRR antagonist polypeptides include but are not limited to IFDNIISQGVLKEDVF (PR10; SEQ ID NO:4), LPTDTTTFKRIFLKRMPSI (PRO20; SEQ ID NO:2), LPTDTTTFKRIFLKRMPSIRE (PR30; SEQ ID NO:5), and LPTRTATFERIPLKKMPSVRE (PR40; SEQ ID NO:6).

In some instances, PRR antagonist polypeptides can comprise an amino acid sequence having at least 70% identity to the amino acid sequence set forth in SEQ ID NO: 2.

In some instances, PRR antagonist polypeptides can comprise the amino acid sequence set forth in SEQ ID NO:2.

In some instances, PRR antagonist polypeptides can consist of the amino acid sequence set forth in SEQ ID NO:2. In some instances, PRR antagonist polypeptides is the amino acid sequence set forth in SEQ ID NO:2.

PRR antagonist polypeptides include modified peptides, e.g., peptides comprising a thioether bridge and/or amino acids that are not standard or naturally occurring in humans, e.g., amino acids found in polypeptides of microbial origin. Examples of non-standard amino acids include, but are not limited to, dehydroalanine (Dha), 2-aminobutyric acid (Abu), and dehydrobutyrine (referred to herein interchangeably as “Dht” or “Dhb”).

Thioether-bridge modified peptides are designed based on the core amino acid sequences of PR10, PR20, PR30, and PR40 in order to avoid peptide degradation by peptidase in vivo. The introduction of one or more thioether bridges makes the resulting peptides more stable and, therefore, strong PRR antagonists. In some examples, the PRR antagonist is one or more thioether-bridge containing peptides, such as those disclosed in U.S. Pat. Nos. 9,573,976 and 9,586,995, each of which is hereby incorporated by reference in its entirety.

PRR antagonist polypeptides can comprise common amino acid substitutions or modifications. In some instances, a PRR antagonist polypeptide derived from the core amino acid sequence of PR20 can comprise amino acid residues 3, 4, 6, 7, and 18 of the amino acid sequence set forth in SEQ ID NO:2. For example, a polypeptide can comprise the amino acid sequence XXTDXTTXXXXXXXXXXSX (SEQ ID NO:1) wherein each of the X's in SEQ ID NO:1 can be any amino acid. For example, SEQ ID NO: 1 comprises 100% identity at amino acids 3, 4, 6, 7, and 18 to amino acids 3, 4, 6, 7, and 18 of SEQ ID NO:2, but could have any amino acid at the other positions. In some instances, a polypeptide can comprise XXTDXTTFXRIXXXXXXSX (SEQ ID NO: 3) wherein each of the X's in SEQ ID NO:3 can be any amino acid. For example, SEQ ID NO:3 comprises 100% identity at amino acids 3, 4, 6, 7, 8, 10, 11, and 18 to amino acids 3, 4, 6, 7, 8, 10, 11, and 18 of SEQ ID NO:2, but could have any amino acid at the other positions.

A. Variants

In some examples, the disclosed methods include administering a PRR antagonist variants or derivatives, such as those disclosed in U.S. Pat. Nos. 9,573,976 and 9,586,995, each of which is hereby incorporated by reference in its entirety. The PRR antagonists can be modified or altered. As used herein, the term “analog” is used interchangeably with “variant” and “derivative.” Variants and derivatives are well understood to those of skill in the art and can involve amino acid sequence modifications. Such, amino acid sequence modifications typically fall into one or more of three classes: substantial; insertional; or deletional variants. Insertions include amino and/or carboxyl terminal fusions as well as intrasequence insertions of single or multiple amino acid residues. Insertions ordinarily are smaller insertions than those of amino or carboxyl terminal fusions, for example, on the order of one to four residues. These variants ordinarily are prepared by site-specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once. Substitutions, deletions, insertions or any combination thereof may be combined to arrive at a final derivative or analog. Substitutional variants are those in which at least one residue has been removed and a different residue inserted in its place. Such substitutions generally are made in accordance with Table 1 and are referred to as conservative substitutions.

Substantial changes in function are made by selecting substitutions that are less conservative than those in Table 1, i.e., selecting residues that differ more significantly in their effect on maintaining (a) the structure of the polypeptide backbone in the area of the substitution, for example as a sheet or helical conformation, (b) the charge or hydrophobicity of the molecule at the target site, or (c) the bulk of the side chain. The substitutions which in general are expected to produce the greatest changes in the protein properties are those in which: (a) the hydrophilic residue, e.g. seryl or threonyl, is substituted for (or by) a hydrophobic residue, e.g., leucyl, isoleucyl, phenylalanyl, valyl or alanyl; Tryptophan, Tyrosinyl (b) a cysteine or proline is substituted for (or by) any other residue; (c) a residue having an electropositive side chain, e.g., lysyl, arginyl, or hystidyl, is substituted for (or by) an electronegative residue, e.g. glutamyl or aspartyl; or (d) a residue having a bulky side chain, e.g., phenylalanine, is substituted for (or by) one not having a side chain, e.g., glycine, in this case, or (e) by increasing the number of sites for sulfation and/or glycosylation.

TABLE 1

| Amino Acid | Non-limiting Exemplary |
|---|---|
| Substitutions | Conservative |
| Original Residue | Substitutions |
| Ala | Ser |
| Arg | Gly; Gln; Lys |
| Asn | Gln; His |
| Asp | Glu |
| Cys | Ser |
| Gln | Asn; Lys |
| Glu | Asp |
| Gly | Ala |
| His | Asn; Gln |
| Ile | Leu: Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

It is understood that one way to define the variants and derivatives of the disclosed proteins herein is to define them in terms of homology/identity to specific known sequences. Specifically disclosed are variants of PRR antagonists herein disclosed which have at least, 70% or at least 75% or at least 80% or at least 85% or at least 90% or at least 95% homology to the PRR antagonists specifically recited herein. Those of skill in the art readily understand how to determine the homology of two proteins.

The polypeptides can be modified by either natural processes, such as posttranslational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, yristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

Variants can also include peptidomimetics. As used herein, “peptidomimetic” means a mimetic of a function of a protein which includes some alteration of the normal peptide chemistry. Peptidomimetics typically are short sequences of amino acids that in biological properties, mimic one or more function(s) of a particular protein. Peptide analogs enhance some property of the original peptide, such as increases stability, increased efficacy, enhanced delivery, increased half-life, etc. Methods of making peptidomimetics based upon a known polypeptide sequence is described, for example, in U.S. Pat. Nos. 5,631,280; 5,612,895; and 5,579,250. Use of peptidomimetics can involve the incorporation of a non-amino acid residue with non-amide linkages at a given position. One embodiment of the present invention is a peptidomimetic wherein the compound has a bond, a peptide backbone or an amino acid component replaced with a suitable mimic. Some non-limiting examples of unnatural amino acids which may be suitable amino acid mimics include β-alanine, L-α-amino butyric acid, L-γ-amino butyric acid, L-α-amino isobutyric acid, L-ε-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

ii. Polynucleotides

Also disclosed is the use of polynucleotides capable of encoding the disclosed polypeptides. Polynucleotide variants of a PRR antagonist are also disclosed. Polynucleotide variants can have substantial identity to a PRR antagonist polynucleotide sequence described herein. A polynucleotide variant can be a polynucleotide comprising at least 70% sequence identity, preferably at least 75%, 80%, 85%, 90%, 95%, 96%, 97%, 98%, or 99% or higher, sequence identity compared to a reference polynucleotide sequence. For example, a reference polynucleotide sequence can be the polynucleotide sequence capable of encoding SEQ ID NO: 2.

iii. Compositions

Disclosed are compositions comprising any of the disclosed polypeptides or polynucleotides.

Disclosed are compositions which can also include a carrier such as a pharmaceutically acceptable carrier. For example, disclosed are pharmaceutical compositions, comprising the peptides disclosed herein, and a pharmaceutically acceptable carrier.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, Pa. 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like.

iv. Vectors

Disclosed are vectors comprising any of the disclosed polypeptides or polynucleotides. Viral and non-viral vectors can be used to administer the disclosed polypeptides or polynucleotides. For example, nanoparticles can be used to delivery any of the disclosed polypeptides or polynucleotides. And viral vectors, such as adenoviral, adeno-associated, and retroviral vectors, can be used to delivery any of the disclosed polynucleotides.

In some instances, the vectors are expression vectors. Thus, expression of polynucleotide of interest within the vector is controlled by the vector. Expression includes, but is not limited to, processes such as transcription, translation, and RNA splicing, if introns are present.

Also disclosed are compositions comprising the disclosed vectors.

6. Administration

In the methods described herein, administration or delivery of the polypeptides, polynucleotides, vectors, or compositions to cells can be via a variety of mechanisms.

Pharmaceutical compositions can be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The following examples are provided to illustrate certain particular features and/or embodiments. These examples should not be construed to limit the invention to the particular features or embodiments described.

### EXAMPLE

This example demonstrates beneficial effects of the (Pro)renin receptor deletion and blockade in high-fat diet induced metabolic syndromes.

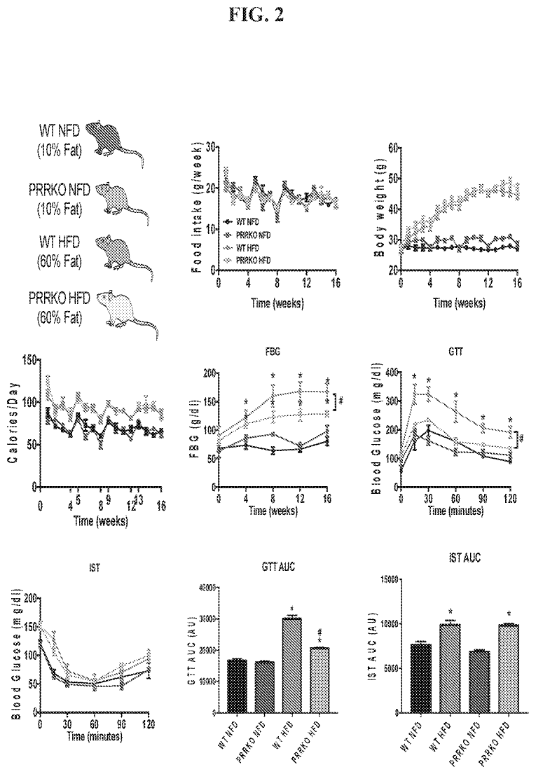

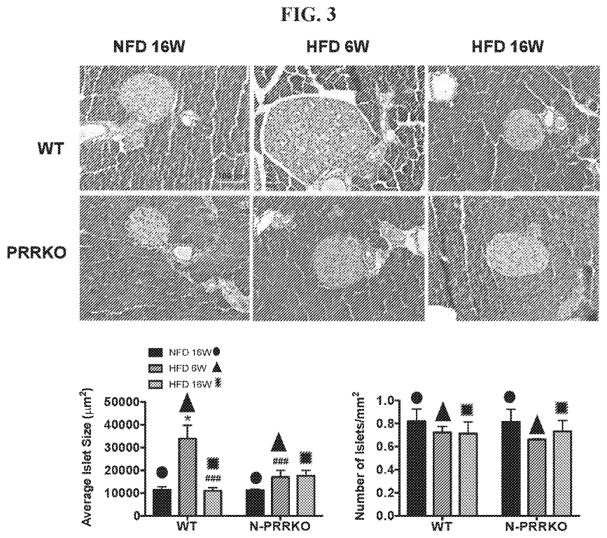

PRR is a key component of the brain renin-angiotensin system, mediating the majority of Ang II formation, and plays a pivotal role in the development of hypertension. Its importance in obesity-related metabolic syndrome is, however, unknown. The inventors believe that brain PRR plays a regulatory role in high-fat diet (HFD) induced metabolic syndrome. To test this hypothesis, neuron-specific PRR knockout (PRRKO) mice and wildtype (WT) littermates were fed with either HFD (60% calories from fat) or normal fat chow (NFD, 10% calories from fat) with matching calories for 16 weeks. Weekly body weight (BW) and monthly fasting blood glucose (FBG) measurements were recorded and end point glucose tolerance (GTT) and insulin sensitivity tests (IST) were performed. Liver samples were taken and paraffin-embedded for histology. Plasma insulin and leptin were measured by ELISA using the UC Davis Mouse Metabolic Phenotyping Core service. Neuronal PRR deletion attenuated the elevation of FBG induced by HFD. Glucose tolerance was significantly improved in PRRKO compared with WT following 16 weeks of HFD, while there was no significant difference in the IST between the groups. Pancreatic islet number and size were evaluated using H&E staining and found that pancreatic islet size was significantly increased following 6 weeks of HFD compared with the in the WT mice, and was reduced to normal size after 16 weeks of HFD. The islet size was significantly smaller in PRRKO compared with WT mice following 6 weeks of HFD. Further, islets in PRRKO mice remained a similar size following 16 weeks of HFD when compared with WT mice in NFD, indicating that PRRKO may protect against islet hypertrophy in HFD. However, no change in islet number was observed among all groups. These data indicate a role of PRR in protecting again HFD-induced islet hypertrophy (see, FIGS. 2 and 3).

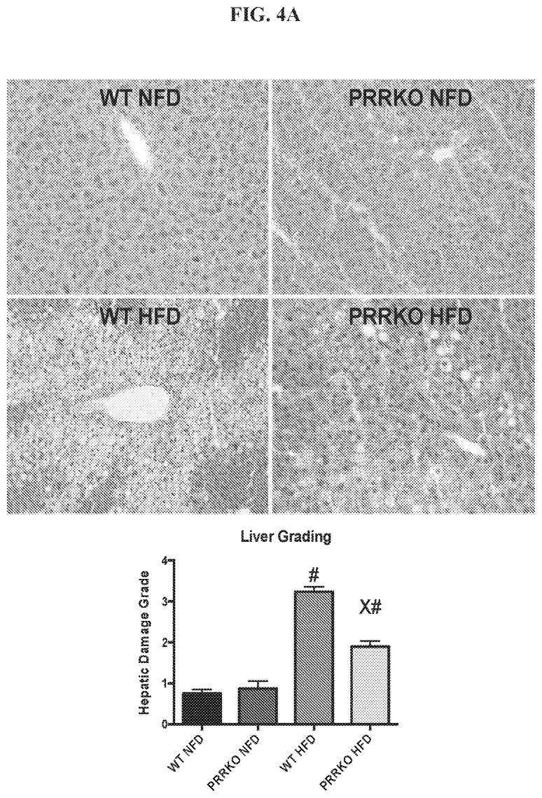

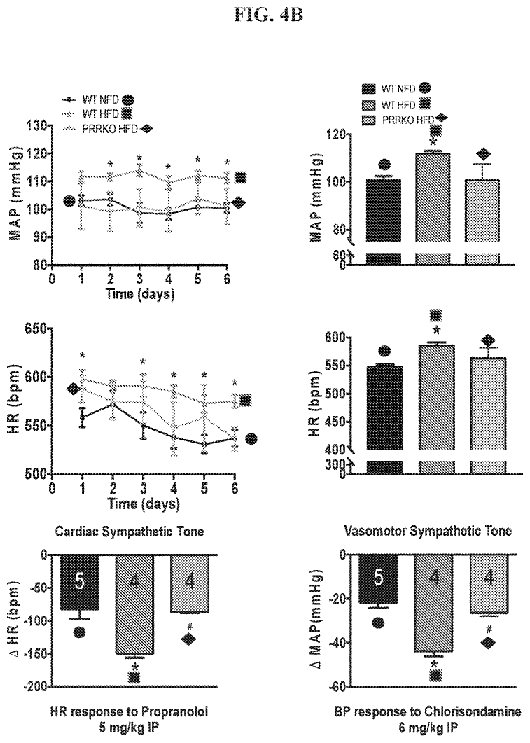

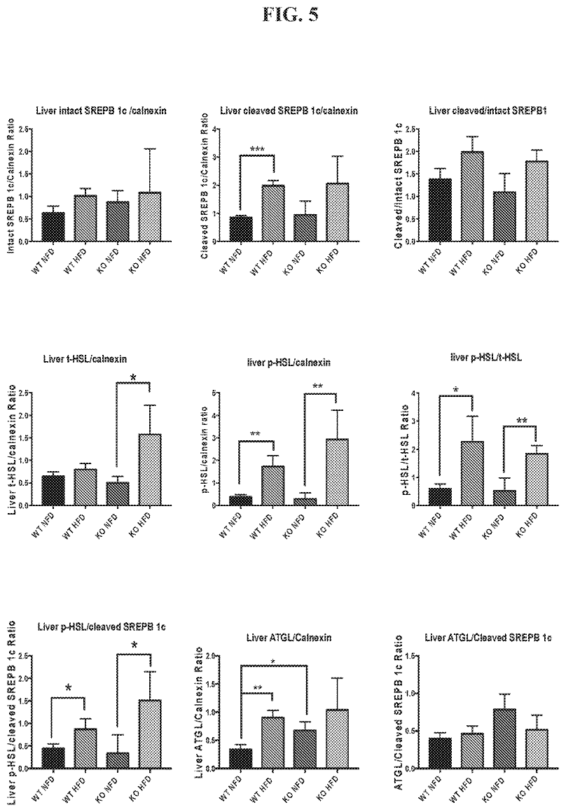

Furthermore, using liver H&E staining, it was found that 16 weeks of HFD induced a significant elevation of liver steatosis in WT mice; while, in the PRRKO mice, the liver steatosis was significantly lower than that of WT mice (FIG. 4A). FIG. 4B illustrates PRR deletion in the neurons reduces high fat diet-induced hypertension. To further understand the molecular mechanisms, western blotting was used to determine the lipid metabolic signaling pathways in the liver. We discovered an elevation of liver lipolysis pathways following PRR deletion. On the contrary, in the WT mice, there was elevation in liver lipogenesis marker following HFD but not in PRRKO (FIG. 5).

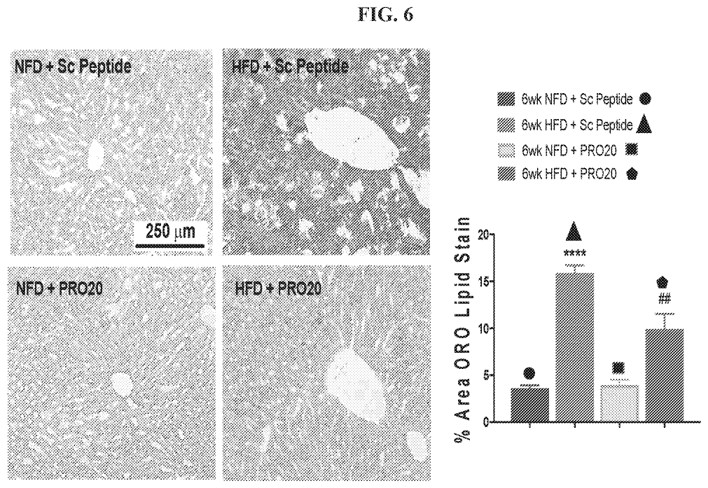

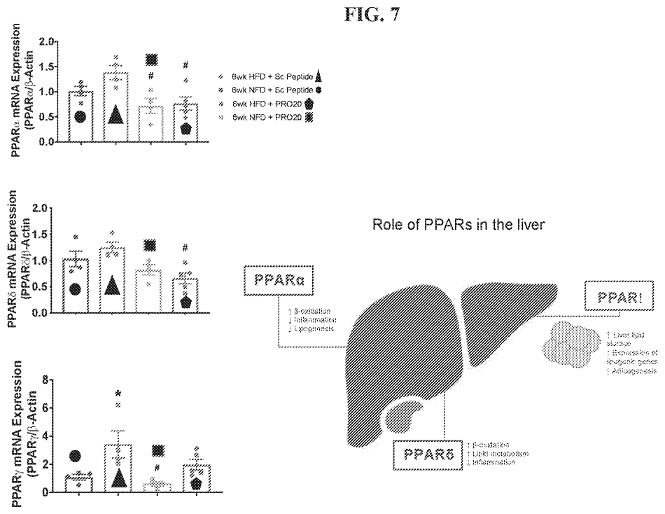

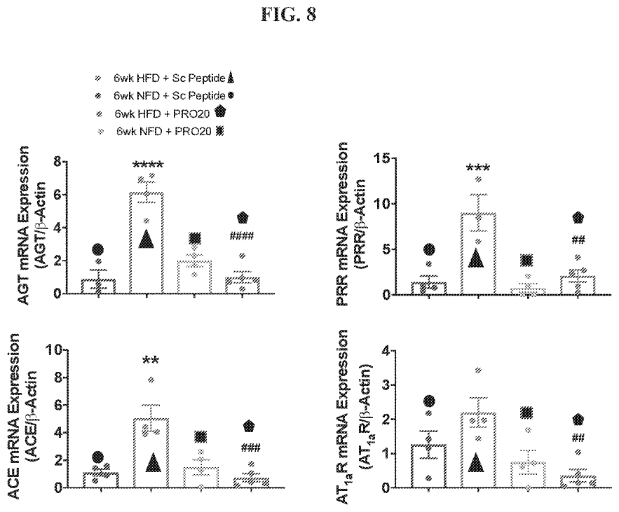

Non-alcoholic fatty liver steatohepatitis (NASH) represents a diverse array of liver damage linked to obesity, metabolic syndrome, and diabetes. The mechanisms and the treatment for NASH remains lacking. The inventors hypothesized that PRR antagonism using PRO20 prevents NASH development. FIG. 6 illustrates subcutaneous administration of PRR antagonist, PRO20, attenuates high fat diet induced-liver steatosis in mice. Wild-type C57Bl/6J mice were treated with either with either HFD (60% calories from fat) or normal fat chow (NFD, 10% calories from fat) with matching calories for 6 weeks. Two week following diet modification, HFD mice were implanted with subcutaneous osmotic pump containing either PRO20 (27 ug/kg/d) or saline for 4 weeks. Liver tissues were processed for Oil Red 0 staining for lipid accumulation and quantified using Image)/FIJI software. Data were presented % of ORO staining to the total area. N=4 mice/group. *p<0.01 vs. NFD; #P<0.05 vs. HFD Saline; One-way ANOVA. 6 weeks of HFD (17.57±3.23%) induced a significant elevation on liver ORO staining compared with the liver from mice fed with NFD (3.51±0.86%, p=0.005). More importantly, PRO20 treatment (7.34±1.96%; p=0.03) significantly reduced the ORO staining in mice treated with HFD. FIG. 7 illustrates subcutaneous administration of PRR antagonist, PRO20, attenuates peroxisome proliferator-activated receptor gamma (PPARγ). FIG. 8 illustrates subcutaneous administration of PRR antagonist, PRO20, prevents activation of liver renin-angiotensin system induced by high fat diet.

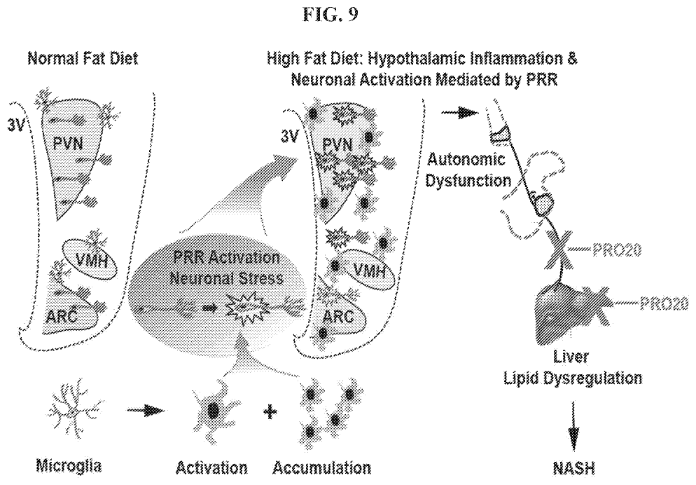

In summary, these findings indicate that PRO20 may be useful for the treatment of high-fat diet related NASH. FIG. 9 provides an exemplary pathway that PRR antagonism reduces high-fat diet induced NASH through dual functions: acting on the central nervous system and directly acting on the liver.

In summary, this example demonstrates that PRR deletion in the neurons attenuates the development of HFD-induced diabetes, protects against glucose intolerance, pancreatic islet hypertrophy and function during HFD possibly by promoting insulin secretion. In addition, PRR blockade attenuates HFD-induced liver steatosis and is associated with the activation of liver lipolysis signal pathways activation. Thus, PRR plays a regulatory role in the development HFD-induced metabolic syndromes. PRR is a novel target for the treatment of liver steatosis and type II diabetes. PRO20 is a PRR antagonist and is a potential therapeutics for the treatment of these metabolic diseases.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. I therefore claim as my invention all that comes within the scope and spirit of these claims.

1. A method of treating, comprising administering to a subject at risk of acquiring or afflicted with non-alcoholic fatty liver disease (NAFLD) an effective amount of a composition comprising a (pro)renin receptor (PRR) antagonist.

2. The method of claim 1, wherein the PRR antagonist is a polypeptide with at least 70% sequence identity to the amino acid sequence set forth in SEQ ID NO: 2, SEQ ID NO: 1, SEQ ID NO: 3, SEQ ID NO: 4, SEQ ID NO: 5, or SEQ ID NO: 6.

3. The method of claim 2, wherein the PRR antagonist is a polypeptide with the amino acid sequence set forth in SEQ ID NO: 2.

4. The method of claim 2, wherein the PRR antagonist is a polypeptide with the amino acid sequence set forth in SEQ ID NO: 5.

5. The method of claim 2, wherein the PRR antagonist is a polypeptide with the amino acid sequence set forth in SEQ ID NO: 6.

6. The method of claim 1, wherein the NAFLD is nonalcoholic steatohepatitis (NASH).

7. The method of claim 1, further comprising selecting a subject at risk or afflicted with NAFLD.

8. The method of claim 1, further comprising diagnosing the subject with NAFLD.

9. The method of claim 1, wherein the composition further comprises a pharmaceutically acceptable carrier.

10. The method of claim 1, wherein the PRR antagonist is administered with an additional therapeutic agent.

11. The method of claim 1, wherein the composition is administered orally, subcutaneously, or intravenously.

12. The method of claim 11, wherein the composition is administered subcutaneously.

13. The method of claim 12, wherein the composition is administered subcutaneously at a dosage of about 200-700 μg/kg/day.

14. The method of claim 1, wherein the subject has Type II diabetes.

15. The method of claim 1, wherein the subject does not have Type II diabetes and/or hypertension.

16. The method of claim 2, wherein the PRR antagonist is a polypeptide with at least 80% sequence identity to the amino acid sequence set forth in SEQ ID NO: 2.

17. The method of claim 2, wherein the PRR antagonist is a polypeptide with at least 90% sequence identity to the amino acid sequence set forth in SEQ ID NO: 2.

18. The method of claim 2, wherein the PRR antagonist is a polypeptide with 100% sequence identity to the amino acid sequence set forth in SEQ ID NO: 2, SEQ ID NO: 1, SEQ ID NO: 3, SEQ ID NO: 4, SEQ ID NO: 5, or SEQ ID NO: 6.

19. The method of claim 1, wherein the effective amount of the composition reduces fasting blood glucose levels in the subject as compared to an absence of administration of the effective amount of the composition.


SEQUENCE LISTING (6 sequences)

SEQ ID NO: 1 - protein, 19 aa, Artificial Sequence
  Synthetic (pro)renin receptor agonist peptide
  misc_feature (1)..(2): Xaa can be any naturally occurring amino acid
  misc_feature (5)..(5): Xaa can be any naturally occurring amino acid
  misc_feature (8)..(17): Xaa can be any naturally occurring amino acid
  misc_feature (19)..(19): Xaa can be any naturally occurring amino acid
XXTDXTTXXXXXXXXXXSX

SEQ ID NO: 2 - protein, 19 aa, Artificial Sequence
  Synthetic (pro)renin receptor agonist peptide
LPTDTTTFKRIFLKRMPSI

SEQ ID NO: 3 - protein, 19 aa, Artificial Sequence
  Synthetic (pro)renin receptor agonist peptide
  misc_feature (1)..(2): Xaa can be any naturally occurring amino acid
  misc_feature (5)..(5): Xaa can be any naturally occurring amino acid
  misc_feature (9)..(9): Xaa can be any naturally occurring amino acid
  misc_feature (12)..(17): Xaa can be any naturally occurring amino acid
  misc_feature (19)..(19): Xaa can be any naturally occurring amino acid
XXTDXTTFXRIXXXXXXSX

SEQ ID NO: 4 - protein, 16 aa, Artificial Sequence
  Synthetic (pro)renin receptor agonist peptide
IFDNIISQGVLKEDVF

SEQ ID NO: 5 - protein, 21 aa, Artificial Sequence
  Synthetic (pro)renin receptor agonist peptide
LPTDTTTFKRIFLKRMPSIRE

SEQ ID NO: 6 - protein, 21 aa, Artificial Sequence
  Synthetic (pro)renin receptor agonist peptide
LPTRTATFERIPLKKMPSVRE